(19) United States
(12) Patent Application Publication
(10) Pub. No.: US 2025/0082678 A1
(43) Pub. Date: Mar. 13, 2025

(54) BIOAVAILABLE MIXTURE PROVIDING SAFE, BROAD-SPECTRUM, ANTIPATHOGENIC, HEALTH, FITNESS, NEUROLOGICAL, AND HOMEOSTATIC BENEFITS

(71) Applicant: Sonnenschein Institute, Inc., Boca Raton, FL (US)

(72) Inventor: Leonard Sonnenschein, Boca Raton, FL (US)

(21) Appl. No.: 18/377,563

(22) PCT Filed: Apr. 8, 2022
(86) PCT No.: PCT/US2022/024096

§ 371 Date: Oct. 6, 2023

Related U.S. Application Data
(60) Provisional application No. 63/173,229, filed on Apr. 9, 2021.

(51) Int. Cl. A61K 33/42; A61K 9/16; A61K 33/00; A61K 33/04; A61K 33/06; A61K 33/08; A61K 33/22; A61K 33/24; A61K 33/26; A61K 33/30; A61K 33/32; A61K 33/34; A61K 33/38; A61K 45/06

(52) U.S. Cl. CPC A61K 33/42; A61K 9/1682; A61K 33/00; A61K 33/04; A61K 33/06; A61K 33/08; A61K 33/22; A61K 33/24; A61K 33/26; A61K 33/30; A61K 33/32; A61K 33/34; A61K 33/38; A61K 45/06

(57) ABSTRACT

An ionic, electrically activated, aqueous or dry mixture includes dissolved minerals, undissolved minerals, and inert, structured, mineral microparticles. The aqueous or dry mixture may be formulated to make the active ingredients systemically bioavailable to safely, conveniently, and effectively achieve broad-spectrum, antipathogenic activity and provide many other health, fitness, neurological, homeostatic, mental, antiaging, cosmetic, veterinary, medical, pharmaceutical, and performance benefits and for methods of treating or managing a disease, condition, or symptom thereof.

### CROSS-REFERENCE TO RELATED APPLICATIONS

This application is a National Stage application and claims priority to, and the benefit of International PCT Application Serial No. PCT/US2022/024096 filed Apr. 8, 2022, and entitled “Bioavailable Mixture Providing Safe, Broad-Spectrum, Antipathogenic, Health, Fitness, Neurological, and Homeostatic Benefits,” which claims priority to, and the benefit of, U.S. Provisional patent application Ser. No. 63/173,229 filed Apr. 9, 2021, and entitled “Bioavailable Mixture Providing Safe, Broad-Spectrum, Antipathogenic, Health, Fitness, Neurological, and Homeostatic Benefits,” the entire disclosures of which are incorporated herein in their entirety by express reference thereto.

### TECHNICAL FIELD

The present disclosure relates generally to an ionic, electrically activated, aqueous or dry mixture comprising dissolved minerals, undissolved minerals, and inert, structured, mineral microparticles. The aqueous or dry mixture may be formulated to make the active ingredients systemically bioavailable to safely, conveniently, and effectively achieve broad-spectrum, antipathogenic activity and provide many other health, fitness, neurological, homeostatic, mental, antiaging, cosmetic, veterinary, medical, and performance benefits. The mixture may also be formulated as pharmaceutical compositions and in methods of treating or managing a disease, disorder, or symptom thereof with such pharmaceutical compositions.

### BACKGROUND

Until now, wellness has been described as a lack of disease; increasingly, however, disease, as evidenced by the existence of pathogens, has become more a state of living with certain pathogens as a matter of consequence. Pathogens can be bacterial, viral, fungal, microalgae, protozoan, and corresponding spores, but to safely eliminate pathogens, specific antipathogen products are typically used with specificity, such as either antibacterial, antiviral, antifungal, or antiprotozoal products. Most of these antipathogens also have some level of toxicity and adverse side effects. There are no known products, other than disinfectants, that can be used to eradicate a wide spectrum of pathogens. Disinfectants cannot be taken internally, and disinfectants like bleach are dangerous to helpful bacteria, healthy cells, and the living body in general. Moreover, most antipathogenic products have particular sites and modes of action which take time and are often ineffective against whole-body infections.

### DETAILED DESCRIPTION

The human body consists primarily of water, which has a similar content to natural seawater. The salts that are dissolved into the cells are constantly being drained by bodily eliminations and may not be fully replaced over time, which can lead to the blood, cells, organs, and even tears being lacking in mineral nutrients. It is important for our own health to replace these needed minerals on a daily basis. Over time, the lack of these necessary minerals can create imbalances, which may create opportunity for pathogen processes to take hold, whether bacterial, viral, fungal, or protozoal in origin.

Not only can taking dietary mineral supplements help to keep you healthy, but also these minerals can aid in strengthening your immune system, improve your energy levels, increase your mental acuity, and help in overall wellbeing. For example, just as calcium builds strong bones, magnesium supports brain functions, boron helps maintain the integrity of cell membranes and activates vitamin D, copper helps in the production of red blood cells and promotes healing and immune functions, chromium helps with energy metabolism, and zinc allows for improved enzyme functioning and immune system bolstering and supports the sense of smell and protein synthesis.

The present disclosure relates generally to an ionic, electrically activated, aqueous or dry mixture comprising a concentrate of dissolved minerals, undissolved minerals, and inert, structured mineral microparticles. This mixture is formulated to make the active ingredients systemically bioavailable to effectively achieve broad-spectrum, antipathogenic activity and provide the body with what it needs to achieve optimal and/or improved physical health and mental health, which includes fitness, strength, speed, mental acuity, beauty, endurance, and athletic performance. This is accomplished with a delivery system that is faster, more convenient, safer, less expensive, more comfortable, less invasive, non-addictive, and in most cases dramatically more effective than any known remedy, including pharmaceuticals, and with no adverse side effects.

In some implementations, as further disclosed herein, the mixture comprises primarily water with trace amounts of safe, dissolved, inert mineral microparticles. Therefore, the only side effects should be beneficial and not adverse. In some implementations, the delivery system of the mixture is by oral ingestion as a non-prescription consumer product, in the form of a dietary supplement drink or a dry powder capsule, providing immune system support or as an energy, fitness, performance, and/or recovery drink or dry powder capsule. In other implementations, the mixture of the present disclosure may be incorporated as an active ingredient and formulated for injectable, inhalable, or topical delivery systems. Some delivery systems for the mixture will require regulatory approval as a medical device, over-the-counter drug, or prescription drug.

The effectiveness of this mixture on humans and animals may, in part, be based on its structured and formulated ability to safely deliver appropriate and precise electrically charged particles and ions to virtually all areas of the body. These electrically charged particles and ions are operable to disable a broad spectrum of current and even future mutated pathogens, potentially halt and reverse many other chronic and disease conditions, provide increased oxygen to tissues throughout the body, reduce inflammation and depression, improve sleep and homeostasis, increase athletic performance, endurance, and recovery, improve mental acuity, and stimulate healing, along with potential antiaging benefits.

Thus, the present disclosure is directed to an ionic, electrically activated, aqueous or dry mixture comprising dissolved minerals, undissolved minerals, and inert structured mineral microparticles, which in combination provide wellness support. The mixture comprises a concentrate, as further described herein, that enhances bioavailability and bioactivity across cell membranes and structures.

### Activated Aqueous or Dry Concentrate Description

In some implementations, the mixture of the present disclosure comprises a concentrate. In some implementations, the concentrate is an activated aqueous or dry concentrate comprising a formulation of minerals, including inorganic, structured, insoluble compounds, and a seawater derivative containing trace elements.

In some implementations, said formulation comprises a mixture of the elements and compounds shown in Table 1 within the identified ranges per serving for a drink, which conform to U.S. Recommended Daily Allowances (RDA) or other generally recognized safe ranges for daily consumption, including Recommended Daily Intake (RDI).

TABLE 1

| | Ingredient | RDA | |
|---|---|---|---|
| | range | or | |
| Element or | Per Serving | RDI* | |
| Compound | mg/oz (30 ml) | mg | Description/Uses |
| Phosphorus | 0.0001 to 0.1 | 700 | A component of bones, cells, in energy |
| | | | processing, in DNA and ATP (as phosphate) |
| Silica, Total | 0.001 to 0.1 | 50* | Silicon is used for weak bones (osteoporosis), |
| (SiO2) | | | heart disease and stroke (cardiovascular |
| | | | disease), Alzheimer's disease, hair loss, and |
| | | | improving hair and nail quality. It is also used |
| | | | for improving skin healing; and for treating |
| | | | sprains and strains, as well as digestive system |
| | | | disorders. |
| Aluminum | 0.01 to 100 | 95* | Aluminum provides health benefits by providing |
| Oxide as | | | a safe barrier to bacteria and contamination in |
| Alumina | | | food preservation. Aluminum |
| | | | compounds increase the effects in vaccines and |
| | | | medicines. |
| Boron | 0.01 to 10 | 13* | Boron is an essential plant nutrient, required |
| | | | primarily for maintaining the integrity of cell |
| | | | walls. In animals, supplemental boron has |
| | | | been shown to reduce calcium excretion and |
| | | | activate vitamin D. |
| Calcium | 0.01 to 100 | 1000 | Needed for muscle, heart, and digestive |
| | | | system health, builds bone, supports |
| | | | synthesis and function of blood cells. |
| Chromium | 0.0001 to 0.020 | 0.035 | Involved in glucose and lipid metabolism, |
| | | | although its mechanisms of action in the body |
| | | | and the amounts needed for optimal health are |
| | | | not well-defined |
| Copper | 0.001 to 0.1 | 0.900 | Required co-factor for cytochrome c oxidase |
| Iron | 0.001 to 5 | 10 | Required for many proteins and enzymes, |
| | | | notably hemoglobin to prevent anemia |
| Magnesium | 0.1 to 200 | 400 | Required for processing ATP and for bones |
| Manganese | 0.0001 to 1 | 2 | Required co-factor for superoxide dismutase |
| Potassium | 0.01 to200 | 2000 | A systemic electrolyte and is essential in |
| | | | coregulating ATP with sodium |
| Silver (1) | 0.05 to 100 | 500* | The medical uses of silver include its use in |
| | | | wound dressings, creams, and as an antibiotic |
| | | | coating. |
| Sodium | 0.01 to 500 | 1500* | Sodium is a systemic electrolyte and is |
| | | | essential in coregulating ATP with potassium |
| Sulfur | 0.1 to1 | 1000* | Sulphur is a main component of keratin a key |
| | | | component of the human skin, nails, and hair. |
| | | | Sulphur is important in the synthesis of collagen, |
| | | | insulin, and can help treat certain types of |
| | | | arthritis. Sulphur is present in glutathione, amino |
| | | | acids, and involved in many enzyme |
| | | | biochemical pathways. |
| Titanium | 0.1 to 1.5 | 10* | Titanium is used from head to toe in biomedical |
| | | | implants. |
| Zinc | 0.1 to 5.5 | 11 | Required for several classes of enzymes such as |
| | | | matrix metalloproteinases, liver alcohol |
| | | | dehydrogenase, carbonic anhydrase, and zinc |
| | | | finger proteins. |

(1) Silver may be substituted with similar elements, such as gold, iron, or copper.

In one implementation, said formulation comprises a mixture of the elements and compounds shown in Table 2 in the identified amounts per serving for a drink, which provide the percentages of U.S. Recommended Daily Allowances (RDA) or (RDI) identified in Table 2.

### Single Serving Example Formulation for Dietary Supplement Drink

TABLE 2

| | Ingredient | RDA | % | |
|---|---|---|---|---|
| | Amount | or | RDA | |
| | Per Serving | RDI* | or % | |
| Element | mg/oz (30 ml) | mg | RDI | Description/Uses |
| Phosphorus | 0.001 | 700 | 0.0001 | A component of bones, cells, in |
| | | | | energy processing, in DNA and |
| | | | | ATP (as phosphate) |
| Silica, Total | 0.093 | 50* | 0.2 | Silicon is used for weak bones |
| (SiO2) | | | | (osteoporosis), heart disease and |
| | | | | stroke (cardiovascular disease), |
| | | | | Alzheimer's disease, hair loss, |
| | | | | and improving hair and nail |
| | | | | quality. It is also used for |
| | | | | improving skin healing; and |
| | | | | for treating sprains and strains, |
| | | | | as well as digestive system |
| | | | | disorders. |
| Aluminum | 9.36 | 95* | 9.9 | Aluminum provides health |
| Oxide as | | | | benefits by providing a safe |
| Alumina | | | | barrier to bacteria and |
| | | | | contamination in food |
| | | | | preservation. Aluminum |
| | | | | compounds increase the effects in |
| | | | | vaccines and medicines. |
| Boron | 0.194 | 13* | 1.5 | Boron is an essential plant |
| | | | | nutrient, required primarily for |
| | | | | maintaining the integrity of cell |
| | | | | walls. In animals, supplemental |
| | | | | boron has been shown to reduce |
| | | | | calcium excretion and activate |
| | | | | vitamin D. |
| Calcium | 0.218 | 1000 | 0.02 | Needed for muscle, heart, |
| | | | | and digestive system health, |
| | | | | builds bone, supports |
| | | | | synthesis and function of |
| | | | | blood cells. |
| Chromium | 0.0002 | 0.035 | 0.57 | Involved in glucose and lipid |
| | | | | metabolism, although its |
| | | | | mechanisms of action in the |
| | | | | body and the amounts needed |
| | | | | for optimal health are not well- |
| | | | | defined |
| Copper | 0.009 | 0.900 | 1.0 | Required co-factor for cytochrome |
| | | | | c oxidase |
| Iron | 0.005 | 10 | 0.005 | Required for many |
| | | | | proteins and enzymes, |
| | | | | notably hemoglobin |
| | | | | to prevent anemia |
| Magnesium | 30.6 | 400 | 7.7 | Required for processing ATP and |
| | | | | for bones |
| Manganese | 0.0002 | 2 | 0.01 | Required co-factor for superoxide |
| | | | | dismutase |
| Potassium | 0.57 | 2000 | 0.03 | A systemic electrolyte and is |
| | | | | essential in coregulating ATP with |
| | | | | sodium |
| Silver(1) | 0.528 | 500* | 0.1 | The medical uses of silver |
| | | | | include its use in wound |
| | | | | dressings, creams, and as an |
| | | | | antibiotic coating. |
| Sodium | 0.861 | 1500* | 0.06 | Sodium is a systemic |
| | | | | electrolyte and is essential in |
| | | | | coregulating ATP with |
| | | | | potassium |
| Sulfur | 2.064 | 1000* | 0.21 | Sulphur is a main component of |
| | | | | keratin a key component of the |
| | | | | human skin, nails, and hair. |
| | | | | Sulphur is important in the |
| | | | | synthesis of collagen, insulin, and |
| | | | | can help treat certain types of |
| | | | | arthritis. Sulphur is present |
| | | | | in glutathione, amino acids, and |
| | | | | involved in many enzyme |
| | | | | biochemical pathways. |
| Titanium | 0.543 | 2* | 27.2 | Titanium is used from head to toe |
| | | | | in biomedical implants. |
| Zinc | 0.504 | 11 | 4.6 | Required for several classes of |
| | | | | enzymes such as matrix |
| | | | | metalloproteinases, liver alcohol |
| | | | | dehydrogenase, carbonic |
| | | | | anhydrase, and zinc finger |
| | | | | proteins. |

(1)Silver may be substituted with similar elements, such as gold, iron, or copper.

In one implementation, said formulation comprises a mixture of the elements and compounds shown in Table 3 in the identified example amounts per 1,500 milligram dose, delivered via one or more dry powder capsules (e.g., two per day), which provide the percentages of U.S. Recommended Daily Allowances (RDA) identified in Table 3.

### Single Dose Example Formulation for Dry Powder Capsules

TABLE 3

| | Ingredient Amount | | % RDA | |
|---|---|---|---|---|
| Element | per 1500 mg Dose | RDA | per Dose | Description/Uses |
| Aluminum | 15 mg | 95 mg | 16 | Aluminum provides health |
| Oxide as | | | | benefits by providing a safe |
| Alumina | | | | barrier to bacteria and |
| | | | | contamination in food |
| | | | | preservation. Aluminum |
| | | | | compounds increase the effects |
| | | | | in vaccines and medicines. |
| | | | | Alumina improves calmness. |
| Calcium | 97 mg | 1000 mg | 10 | Needed for muscle, heart, |
| | | | | and digestive system |
| | | | | health, builds bone, |
| | | | | supports synthesis and |
| | | | | function of blood cells. |
| | | | | Calcium benefits bones |
| | | | | and nerves. |
| Chromium | 5 mcg | 50 mcg | 6 | Involved in glucose and lipid |
| | | | | metabolism, although its |
| | | | | mechanisms of action in the |
| | | | | body and the amounts needed |
| | | | | for optimal health are |
| | | | | not well-defined. Chromium |
| | | | | increases protein and |
| | | | | carbohydrate metabolism. |
| Copper | 0.05 mg | 0.5 mg | 10 | Required co-factor for |
| | | | | cytochrome c oxidase. Copper |
| | | | | increases nerve and immune |
| | | | | benefits. |
| Iodine | 21 mcg | 150 mcg | 15 | Iodine helps hormones. |
| Iron | 1.6 mg | 18 mg | 10 | Required for many |
| | | | | proteins and enzymes, |
| | | | | notably hemoglobin |
| | | | | to prevent anemia. Iron |
| | | | | increases immunity and |
| | | | | improves energy. |
| Magnesium | 45 mg | 400 mg | 11 | Required for processing ATP and |
| | | | | for bones. Magnesium increases |
| | | | | muscles and improves nerve |
| | | | | conditions. |
| Manganese | 0.45 mg | 2 mg | 23 | Required co-factor for superoxide |
| | | | | dismutase. Manganese improves |
| | | | | connective tissue. |
| Molybdenum | 5 mcg | 50 mcg | 10 | Molybdenum decreases |
| | | | | inflammation. |
| Selenium | 5 mcg | 50 mcg | 10 | Selenium decreases |
| | | | | inflammation. |
| Titanium | 1.1 mg | 2 mg | 55 | Titanium is used from head to |
| | | | | toe in biomedical implants. |
| | | | | Titanium regulates body energy. |
| Vanadium | 2 mcg | 22 mcg | 10 | Vanadium increases athletic |
| | | | | performance. |
| Zinc | 15 mg | 11 mg | 136 | Required for several classes of |
| | | | | enzymes such as matrix |
| | | | | metalloproteinases, liver alcohol |
| | | | | dehydrogenase, carbonic |
| | | | | anhydrase, and zinc finger |
| | | | | proteins. Zinc supports the |
| | | | | immune system. |

In some implementations, said formulations comprise insoluble, structured, mineral microparticles consisting of a very thin surface layer of titanium oxide, covalently bonded to a high porosity ceramic substrate, with metal or metal salt crystalized structures featuring sharp edges and/or points covalently bonded to at least part of the titanium oxide surface layer, which together yield a net-positive electrical charge. In some implementations, the ceramic substrate is alumina. In some implementations, the ceramic substrate comprises alumina, silica, zirconia, calcia, magnesia, ceria, sodia, potassia, lithia, boron oxide, manganese oxide, iron oxide, cobalt oxide, zinc oxide, or any combination thereof. In some implementations, the metal or metal salt is silver chloride. In some implementations, the metal or metal salt comprises silver, gold, iron, copper, platinum, palladium, cobalt, tin, zinc, zirconium, molybdenum, or any combination thereof, or any salt thereof. In some implementations, the metal or metal salt includes zinc. In some implementations, the ceramic substrate excludes alumina. In some implementations, the metal or metal salt excludes a salt. In some implementations, a weight ratio of the ceramic substrate to the titanium dioxide is from about 5:1 to 20:1, about 5:1 to 10:1, or about 10:1 to 15:1. In some implementations, a weight ratio of the ceramic substrate to the metal or metal salt is from about 5:1 to 20:1, about 5:1 to 10:1, or about 10:1 to 15:1.

In some implementations, the ceramic substrate is activated or surface modified to include active groups on a porous surface thereof. In some implementations, the active groups are hydroxyl groups that are distributed over and associated with the porous surface. In one implementation, these hydroxyl groups are uniformly distributed while in another they are non-uniformly distributed. In some implementations, the titanium dioxide and/or the metal or metal salt may be bound to the ceramic substrate via the active groups. In some implementations, the structured particle includes exposed active groups that are not associated with the titanium dioxide or the metal or metal salt. In some implementations, the titanium dioxide forms a discontinuous layer on the ceramic substrate.

In some implementations, the seawater derivative of said concentrate comprises 1.0 milliliter per single serving or up to 1,500 milligrams per single dose, which provides, in addition to those elements and compounds listed in Tables 1, 2 & 3, trace elements in minute quantities, which may include: Selenium, Vanadium, Molybdenum, Lithium, Carbon, Chlorine, Oxygen, Nitrogen, Hydrogen, and Bromine. These trace elements are only added as part of natural seawater derivatives at extreme trace quantities, which are unmeasurable due to the limits of commercial laboratory equipment specifications.

In some implementations, the dry powder formulation, in addition to those elements and compounds listed in Table 3, comprises trace elements in minute quantities, which may include: Alfalfa, Boron, Chamomile, Dandelion Root, Glutamic Acid, Horsetail, Parsley, Phosphorous, Potassium, Rice, Silver (or similar elements, such as Gold, Iron, or Copper), Watercress, and Yellow Dock Root. These trace elements are only added at extreme trace quantities, which are unmeasurable due to the limits of commercial laboratory equipment specifications. A dry powder formulation provided in capsule, tablet or caplet format may include rice or other excipients.

In some implementations, at least some of the component ingredients of said concentrate are electrically active. In other implementations, at least some of the component ingredients are electrically activated by a proprietary process. In some implementations, water is one of the component ingredients that is electrically active. In some implementations, the electrically active water is “Treated Energized Water (TEW)” formed by a process that treats water with an electromagnetic field to increase the water's electrical conductivity to at least about 500 μS/cm, at least about 800 μS/cm, at least about 1,000 μS/cm, or at least about 2,000 μS/cm, each at 25° C. In some implementations, the TEW treatment process increases the water's electrical conductivity by at least about 30%, at least about 40%, at least about 50%, at least about 60%, at least about 70%, at least about 80%, at least about 90%, at least about 100%, about 30 to 100%, about 40 to 90%, or about 50 to 80%.

In some implementations, the mixture includes the structured particles and a pharmaceutically acceptable additive. In some implementations, the additive is magnesium. In some implementations, the additive comprises phosphorous, silica, alumina, boron, calcium, chromium, copper, iron, magnesium, manganese, potassium, gold, silver, sodium, sulfur, titanium, zinc, or any combination thereof. In some implementations, a weight ratio of the structured particles to the additive in the mixture is from about 10:1 to 1:100, about 5:1 to 1:15, about 1:1 to 1:20, about 5:1 to 1:10, or about 1:1 to 1:2. In some implementations, the additive comprises magnesium and a weight ratio of the structure particles to magnesium is from about 10:1 to 1:20, about 2:1 to 1:10, about 2:1 to 1:5, or about 1:1 to 1:2.

### Method of Forming Activated Aqueous or Dry Concentrate

The method of forming the activated aqueous concentrate of the present disclosure comprises the steps of preparing said formulation of powdered elements and compounds and adding said liquid seawater derivative.

In some implementations, the method of preparing said formulation comprises micronizing beads, granules, or other structures comprising the elements and compounds within the ranges listed in Table 1 or Table 3 into a powder. In various implementations, the micronizing produces a powder having particles in the range of 100 nanometers to 450 nanometers and/or particles in the range of 100 microns to 450 microns. The micronizing may be accomplished by grinding or other processes well known in the art.

In other implementations, the method of preparing said formulation comprises creating a mixture of the elements and compounds within the ranges listed in Table 1 or Table 3 and may include micronizing the mixture into a powder having particles in the range of 100 nanometers to 450 nanometers and/or particles in the range of 100 microns to 450 microns. The micronizing may be accomplished by grinding or other processes well known in the art.

The method of preparing said formulation may include a method of producing insoluble, structured, mineral microparticles sized to be bioavailable, yet not considered a nanoparticle, which is defined as being smaller than 100 microns. The method of preparing said formulation may include a method of producing insoluble, structured, mineral microparticles sized to be bioavailable, and also considered to be a nanoparticle. In some implementations, the method of producing said bioavailable mineral microparticles results in microparticles in the range of 100 nanometers to 300 nanometers, microparticles of less than about 0.1 microns, and/or microparticles in the range of about 0.1 microns to 300 microns, which are absorbable by the oral mucosa and digestive tract into the circulatory system.

The method of preparing said formulation may further comprise producing structured, mineral microparticles that are larger than 300 microns. These larger structured, mineral microparticles, alone or in combination with bioavailable mineral microparticles (in the range of 100 nanometers to 300 nanometers, less than about 0.1 microns, and/or in the range of about 0.1 microns to 300 microns), that are not absorbed into the circulatory system, provide effective, adjunctive, therapeutic, broad-spectrum, anti-pathogenic activity inside the oral cavity and gastrointestinal tract.

The method of producing said bioavailable mineral microparticles and said larger structured, mineral microparticles may comprise micronizing larger structures, up to about 10,000 times larger than the microparticle size range required for bioavailability for absorption into the circulatory system. Such a micronizing step creates mineral microparticles having more than 100,000 times more surface area per gram than the original larger structures and creates by orders of magnitude more sharp edges and points where electric charges concentrate. In some implementations, before micronization the larger structures may have an average particle size of about 3,000 microns, greater than about 1,000 microns, about 1,000 to about 3,000 microns, or about 2,000 to about 5,000 microns. In some implementations, the larger structures are made by forming a continuous layer of titanium dioxide on a surface of a ceramic particle having an average particle size of about 3,000 microns, greater than about 1,000 microns, about 1,000 to about 3,000 microns, or about 2,000 to about 5,000 microns and then binding a metal or metal salt to at least a portion of the titanium dioxide. In one implementation, the metal or metal salt excludes silver, in another implementation the ceramic excludes alumina, and in a third implementation both silver and alumina are excluded from the larger structures.

The method for forming the activated aqueous or dry concentrate may further comprise mixing said formulation with the seawater derivative in the proportions of 100 mg to 1,000 mg of said formulation with 1.0 milliliter or up to 1,500 milligrams of the seawater derivative. This step of the method infuses the concentrate with electrically charged ionic trace elements that are present in the seawater derivative in minute quantities.

The method for forming the activated aqueous or dry concentrate may further comprise forming TEW by treating water with an electromagnetic field (rather than with minerals) to increase the conductivity of the water to at least about 500 μS/cm, at least about 800 μS/cm, at least about 1,000 μS/cm, or at least about 2,000 μS/cm, each at 25° C. In some implementations, the TEW treatment process increases the water's electrical conductivity by at least about 30%, at least about 40%, at least about 50%, at least about 60%, at least about 70%, at least about 80%, at least about 90%, at least about 100%, about 30 to 100%, about 40 to 90%, or about 50 to 80%. The process of forming TEW comprises use of a proprietary device that concentrates an electromagnetic spectrum of energy to the water.

### Aqueous or Dry Mixture Description

The ionic, electrically activated, aqueous or dry mixture of the present disclosure may comprise the activated aqueous or dry concentrate described above. In one implementation, the mixture of the present disclosure consists only of said activated aqueous or dry concentrate. In another implementation, the mixture of the present disclosure consists of said activated aqueous or dry concentrate in diluted form. In other implementations, the mixture of the present disclosure consists of said activated aqueous or dry concentrate, in original or in diluted form, with other added ingredients.

In various implementations, the ionic, electrically activated, aqueous or dry mixture of the present disclosure may be formulated for a variety of different delivery systems, including, but not limited to: drinks, ingestible products, injections, inhalers, respirators, nebulizers, pills, capsules infusions, topical applications, and in other forms. The mixture of the present disclosure may be formulated, with the same effective ingredients and with the necessary attributes to qualify for a variety of market and regulatory channels, including, but not limited to: a homeopathic product; an over-the-counter medication; a prescription medication; a medical device; a pharmaceutical device; a dietary supplement; a food product; a drink product, including, but not limited to, a sports drink, a fitness drink, a health drink, and an energy drink; a cosmetic product; a personal care product; a nasal spray; a veterinary product; a pet food product; and a livestock product.

In one implementation, the mixture of the present disclosure may be formulated as a drinkable dietary supplement by diluting 1.0 milliliter of the concentrate described in Table 2 above with 30 milliliters of drinking quality, spring water (with accompanying trace amounts of naturally dissolved minerals, not additives), to create a one-ounce single serving size of the dietary supplement in drink form. In some implementations, a daily dose of dietary supplement in drink form comprises the one-ounce single serving size and 100 mg to 1,000 mg of guar gum or similar product to stabilize and thicken the dietary supplement drink.

One mixture of the present disclosure, formulated for use as a drinkable dietary supplement, comprises 99.996% water by weight with 0.002% alumina, 0.001% zinc, and 0.001% all other ingredients as extracted from natural seawater, as listed above by weight in Table 1. The mixture should have a pH level between 6.0 and 8.0 and no measurable microbiological activity.

In one implementation, the mixture of the present disclosure may be formulated as a dry powder by diluting 1,500 milligrams of the concentrate described in Table 3 above with 1-10 milligrams of rice powder infused with dehydrated electrically activated water. Such rice powder may be formed by mixing 1.5 liters of electrically activated water with 1,000 grams of rice powder, and heating the mixture until dry to infuse the rice powder with dehydrated electrically activated water. In some implementations, the electrically activated water is TEW.

The formulated mixture of the present disclosure is affordable, compact, self-sanitizing, requires no refrigeration, and has a multi-year shelf life. The formulated mixture of the present disclosure comprises Generally Recognized as Safe (GRAS) and FDA Approved Food Additives, is safe for endogenous and exogenous use, safe for daily use, easily absorbed, bioactive, nontoxic, digestible, non-corrosive, and residue-free.

In some implementations, the mixture of the present disclosure may be formulated to include higher mineral concentrations, in the range of 3 to 20 times that of the drinkable dietary supplement for daily immune support described above, for interventional applications to address an imminently threatening or existing disease condition. Higher concentrations do not approach any safety level concerns for the minute quantity of the GRAS minerals used in the mixture of the present disclosure. No adverse effects have been reported for more than 100,000+ evaluation samples for both the drinkable and dry capsule products that have been distributed, to date, mostly in the higher mineral concentration range, nor does the science, art, or literature for these ingredients indicate any safety issues in the amounts used in the mixture of the present disclosure.

In some implementations, at least some of the component ingredients of said mixture are electrically active. In other implementations, at least some of the component ingredients are electrically activated by a proprietary process.

### Mechanisms of Action

The ionic, electrically activated, aqueous or dry mixture of the present disclosure may be delivered systemically or topically to treat, cure, heal, prevent and/or mitigate a broad spectrum of human and animal diseases, disorders, and conditions. The mixture of the present disclosure comprises a nontoxic solution made of specific elements and compounds, some of which carry an electrical charge having a propensity to cause certain mechanisms of action to occur.

As further described herein, the mixture may, at appropriate strength, polarity, duration, proximity, and intensity, deliver with therapeutic effect at the cellular and microbial level at least some of the following: an electric charge, an electric field, a related electromagnetic field, an electrostatic force, and an electrical current discharge.

As previously disclosed herein, the concentrate and, therefore, the formulated mixture of the present disclosure, include structured mineral microparticles comprising metal salts and oxides that have been fused into an inert structure consisting of ceramic and crystal elements. These structured microparticles are insoluble in water or body fluids, including stomach acid, and are primarily structured and designed to deliver and maintain a concentrated positive electrical charge at the edges and points of their surface crystalline features.

Minerals, such as silver or other similar halides, aluminum oxides, and titanium oxides are used in this structured microparticle. Each of these mineral components is specifically structured to optimize the generation and maintenance of a strong positive charge at the points and edges of the crystalline surface features. The strong positive charge is generated and maintained because the attached titanium oxide substrate and the attached alumina ceramic substrate constantly drain electrons, which have a negative charge, from the attached silver chloride or other similar halides crystalized structure. The drained electrons are dissipated into the environment (body fluids) over orders of magnitude greater surface area than the strong, concentrated, positive charge at the sharp points and edges of the silver chloride or other similar halides crystalized structure. This allows the structured, mineral microparticles to maintain their antipathogenic capability.

The structured microparticles are formed by combining the three minerals identified in paragraph in three layers. The second layer provides a perfect surface to fuse the third, outer layer of minerals, which are crystalized by a specific process, to create multiple edged and pointed (sharp) crystal surface structure at various locations on the second layer.

Pathogens, without known exceptions, have a net-negative charge, while healthy cells have a net-positive or neutral charge. Since the structured mineral microparticles of the present disclosure carry and maintain an amplified and concentrated positive charge on their sharp surface features, an electrostatic force is created, which draws and holds the pathogens and the microparticles together. For the same reason, the structured mineral microparticles are more likely to be absorbed through the oral mucosa and gastrointestinal tract, which have weak negative charges on their mucus surface coatings, and those weak negative charges interfere with like-charge (i.e., negative charge) particle absorption.

Pathogens are drawn to the structured microparticle surfaces by opposite electrical charges and those pathogens are then fragmented by the electrical charges or by the crystalline structure of the microparticles or both. In more detail, when the pathogens and the structured microparticles are drawn together by opposite charges, the sharp crystalized features of the structured microparticles can both physically damage or disable a pathogen by exerting a disruptive force on a pathogen's external membrane or capsid and/or disable the pathogen when the structured microparticle and pathogen are drawn together at the point of maximum electric potential difference and the structured microparticle delivers an electric discharge or electrostatic force across the fragile pathogen membrane or capsid. This causes the membrane or capsid to leak or rupture, disabling the pathogen's ability to replicate. Pathogen membranes or capsids are under several atmospheres of internal pressure, in some cases 10 to 20 atmospheres. This internal pressure contributes to the catamorphic fragmentation of the pathogen when the membrane or capsid is weakened or breached.

Exogenously, scanning electron microscope images show the pathogens being fragmented by the electrical discharge between the negatively charged pathogens and the positively charged surface features of the structured microparticles eliciting electrical discharges and or mechanical pathogen surface disruptions. These fragmented pathogens could possibly contribute to continuity of immunity enhancement. Though harmless to the body, the adaptive immune system may see these fragments as invaders and begin the process of producing antibodies specific to that pathogen. Thus, the pathogen fragments may remain in the body long enough for the adaptive immune system to begin to make antibodies to prevent future infection. The disabling action of the structured microparticles may create an endogenous vaccine, as well as an increased capacity for charge concentration points, whereby electrostatic force can pull this structured microparticle, with positively charged, sharp crystalline features, together with net-negatively charged pathogens.

Thus, several mechanisms for disabling the pathogen are simultaneous employed by the structured microparticle. Its unique crystalline surface features amplify the intensity of the positive electrical charge concentrated at the points and edges. This creates a stronger electrostatic force, pulling and holding the pathogens and the structured microparticle together. Under the electrostatic force, the sharp features of the silver halide surface structure may damage the pathogen membrane or capsid, causing leakage or damage to the interior contents which are often packed under pressure behind a thin membrane, with ionic transport channels that could be shut down by the interference of the microparticle's strong electric field. Electrical charges can be potentiated for greater-than-normal voltage concentration and discharge, additionally aiding in the surface disruptions of pathogens that come into contact or near-contact with the microparticles, resulting in disruption of the pathogen membrane or capsid, thereby destroying the potency of the pathogens.

The pathogen membrane, cell wall or capsid can be breached by forced close proximity to the structured microparticle's sharp features for any or all of the following reasons: the membrane, cell wall, or capsid may be pierced by sharp particle features, and/or by electrical discharge between the strongest points of voltage differential, and/or by particle electric field interference with the pathogen's ionic channels, and/or other vital, electrically mediated pathogen process. The intense charges at the structured microparticle's peaks and edges may create small, local amounts of H₂O₂ (hydrogen peroxide) or HCLO (hypochlorous acid). Both H₂O₂ and HCLO are produced by the body's innate immune system to disable a broad spectrum of invading pathogens. The structured particle and TEW can break H₂O hydrogen bonds and create H₂O₂ and HCLO similar to the anti-pathogens used by the body's innate immune cells. In general, this process happens very quickly, usually beginning within minutes of ingestion or topical application of the formulated mixture of the present disclosure.

These charged points may potentially create a combined electromagnetic force, further aiding pathogen interaction by electrostatic force to the structured mineral microparticle, the physical interaction described in paragraph [045], and ionic solution potentials for antipathogenic capacity.

For regulatory purposes, the structured mineral microparticle may be considered a medical device for counteracting the effects of viruses, bacteria, and pathogens in the blood and blood components.

The elements of this structured mineral microparticle are considered safe for ingestion, used in minute quantities, and are fused into an inert structure so that these elements cannot individually interact with healthy tissues or organs. After the user has experienced the benefits from the formulated mixture, the structured microparticles are filtered out of the lymphatic and circulatory system by the liver and kidneys and excreted through the urinary and gastrointestinal system and normally digested as well, during the days following ingestion. This process probably eliminates any existing kidney or urinary tract infection as an added benefit.

Other minerals, such as zinc, boron, magnesium, are activated and included in the formulated mixture of the present disclosure. Their function is to enhance bioavailability and supplement the important minerals needed for many essential bodily functions, as is known to those familiar with the art. The mixture of the present disclosure, when formulated for use as a drinkable dietary supplement, contains electrically charged water molecules and their derivative electrically active ions, as well as the natural minerals dissolved in commercially available, drinking quality spring water that may be used to dilute the concentrate of the present disclosure.

The TEW and the activated minerals (i.e., zinc, boron, magnesium and more), which may be seawater derivatives, can break the hydrogen-oxygen valence bond and create a measurable cascade of oxygen freed from the H₂O molecules. For example, when 1 milliliter of the concentrate of the present disclosure is mixed with 30 milliliters of drinking quality spring water to produce a 31-milliliter mixture of the present disclosure in drinkable dietary supplement form, the measurable O₂ content of the mixture is approximately doubled. This cascade release of free O₂ from H₂O molecules continues measurably in the blood stream of a person drinking the electrically energized mixed 31-milliliter dietary supplement. This increase in blood oxygen is evidenced within minutes of oral ingestion and absorption into the blood stream, through the oral mucosa and digestive tract, by recording blood oxygen readings on an oximeter.

Using ionic forms of minerals derived from natural seawater, these dissolved and undissolved substances aggregate free radical charges to create a mixture that is easily absorbed and can, in turn, accelerate other physiologic actions that can promote rapid healing. The mucus lining of the oral mucosa and digestive track is negatively charged and may be an important first entry point for the mixture's action. The structured mineral microparticles of the mixture carry a strong positive charge. Research shows this will aid in absorption into the blood stream through those membranes of larger particles. In some implementations, the microparticles are sized between 100 microns and 300 microns for sublingual absorption into the blood stream. Microparticles sized down to about 0.1 microns have also been shown to be effective and are encompassed within this disclosure. The mineral microparticle is not degraded in the stomach and is absorbed readily in the gastrointestinal tract. The mixture achieves fast systemic availability and effectiveness throughout the circulatory system and the tissues and organs it services.

### Mixture of the Present Disclosure Formulated and Tested as a Dietary Supplement

Recent supervised, independent, clinical studies based on use of the mixture of the present disclosure formulated as a drinkable dietary supplement have shown improvement in dissolved blood oxygen, improvement in body temperature regulation, decreases in clinical depression and anxiety, disappearance of asthma and lung impairment symptoms, decreases in arthritis and pain complaints, improvements in mental acuity, increased energy, improvements in wellness, and better sleep.

A secondary benefit of the aqueous mixture of present disclosure is a significant, measurable release of free oxygen in the bloodstream, creating an immediate sense of wellness and enhanced athletic performance, endurance, recovery, and healing.

In vitro studies of the aqueous mixture of the present disclosure have shown increases in oxygen content, electrical conductivity, and antipathogenic activity.

Additional indications of use for the ionic, electrically activated, aqueous or dry mixture of the present disclosure, derived from research, science, and anecdotal reports, and subject to required clinical studies and regulatory approval of claims, include treatment of: leaky gut syndrome; Lyme disease; urinary tract infections; autoimmune diseases; COVID-19; influenzas; surgical wounds, and other areas of infection caused by injury; infections of the blood, such as Staphylococcus and MERSA; the common cold; antiaging markers; neurodegenerative diseases; and many forms of cancer (cancer cells generally have a negative surface charge vs. positive-to-neutral charge for healthy cells). In general, chronic inflammation and other seemingly irrevocable conditions have, by anecdotal report and theory, been transformed to acute infections with new healing that occurs by utilizing the ionic, electrically activated, aqueous or dry mixture of the present disclosure, indicating a novel electrical/electrochemical mechanism to initiate and sustain healing. The mixture of the present disclosure may have longer lasting or permanent benefits (i.e., as a cure rather than a treatment or palliative measure), as compared to the best available treatments currently approved for many diseases, disorders, and conditions in humans and in animals. The mixture of the present disclosure will accomplish this novel approach to wellness with safe, inorganic, mineral ingredients designed to attack pathogens and diseases the way the body's innate immune system does, electrically and electrochemically, and not with artificial organic molecules, nano-particles, toxic substances, genetic material, or radioactive material, or their related side effects and general ineffectiveness.

Underlying mechanisms of action stimulated by use of the ionic, electrically activated, aqueous or dry mixture of the present disclosure further include: modulation of the cytokine inflammatory response; stimulation of neutrophil macrophage, utilizing hypochlorous acid derived from the active oxygenation from the aqueous or dry mixture of the present disclosure in combination with the body's natural immune system; benefits of increased oxygenation of the blood; and increases in immune responsiveness as measured by higher volumes of antibodies and length of antigen-antibody responsiveness.

Sites of action for the ionic, electrically activated, aqueous or dry mixture of present disclosure include, but are not limited to, the mouth, the esophagus, the stomach, the large and small intestines, and the blood. Additional sites recognized for absorptive actions include the brain, the lungs, the kidneys, the urinary tract, the liver, and the connective tissues, including the skin.

Accordingly, in one aspect, the present disclosure comprises an aqueous and mineral mixture as described herein.

In another aspect, the present disclosure comprises an ionic, electrically activated mixture of water and trace quantities of soluble and insoluble minerals, which provides safe bioavailable, broad-spectrum, antipathogen activity, enhanced health, homeostatic, neurological, longevity and fitness benefits, as disclosed herein.

In another aspect, the present disclosure comprises a mixture including 90% or more by volume drinking-quality, spring water plus accompanying trace amounts of naturally dissolved minerals, not additives, as disclosed herein. The mixture may further comprise soluble and insoluble seawater minerals, including zinc, magnesium, copper, calcium, and trace amounts of other minerals naturally occurring in seawater, as disclosed herein.

The mixture may further comprise structured, mineral microparticles, as disclosed herein. The structured, mineral microparticles are nontoxic, as disclosed herein. The structured, mineral microparticles are insoluble in water and bodily fluids, including stomach acid, as disclosed herein. The structured, mineral microparticles may consist of a very thin layer of titanium oxide, covalently bonded to a high-porosity alumina ceramic substrate, with silver chloride crystalized structures featuring sharp edges and/or points, as disclosed herein. These crystalized structures are covalently bonded to all or parts of the titanium oxide surface layer, which together yield a net-positive electrical charge, as disclosed herein. The attached titanium oxide substrate and the attached alumina ceramic substrate constantly drain electrons, which have negative charges, from the attached silver chloride crystalized structure, as disclosed herein. The drained electrons are dissipated into the environment (body fluids) over orders of magnitude greater surface area than the strong, concentrated, positive charge at the sharp points and edges of the crystalized structures, which allows the structured, mineral microparticles to maintain their antipathogenic capability, as disclosed herein. The structured, mineral microparticles are operable to sustain and concentrate a strong positive charge at the sharp edges and points of the crystalized structures, which creates attraction, by electrostatic force, to move the structured, mineral microparticles into contact with or into close proximity to negatively charged pathogens, as disclosed herein. The strong positive charge may also increase the likelihood the structured, mineral microparticles will be absorbed through the oral mucosa and gastrointestinal tract, which have mucus surfaces with weak negative charges that interfere with like-charge (i.e., negative charge) particle absorption, as disclosed herein.

In yet another aspect, the present disclosure comprises a method of producing structured, mineral microparticles, as disclosed herein. The method may comprise producing bioavailable, structured, mineral microparticles sized between 100 microns and 300 microns and structured, mineral microparticles larger than 300 microns, as disclosed herein. The method of producing structured, mineral microparticles may comprise micronizing structures approximately 10,000 times larger than the resulting microparticles, as disclosed herein. The micronizing step may create microparticles having more than 100,000 times more surface area per gram than the original structures, as disclosed herein. The micronizing step may further create significantly more sharp edges and points to concentrate static electric charges, as disclosed herein.

In still another aspect, at least some of the component ingredients of the mixture of the present disclosure are electrically active, or at least some of the component ingredients of the mixture are electrically activated by a proprietary process.

In yet another aspect, the present disclosure is directed to a method of forming a mixture, as disclosed herein. The mixture may be nontoxic and/or bioavailable, as disclosed herein.

### Pharmaceutical Composition

The present disclosure also provides a pharmaceutical composition including the mixture described herein in therapeutically effective amounts and a pharmaceutically acceptable carrier. In the context of the pharmaceutical composition, the “mixture” refers to the structured particles described herein and one or more added minerals, as described above.

Methods of treatment described herein include treating or managing a disease, condition, or disorder, or a symptom thereof, by administering to an individual in need thereof a therapeutically effective amount of the mixture to treat or manage the disease, condition, or disorder, or one or more symptoms thereof. The phrase “therapeutically” in connection with the effective amount includes that amount of the mixture that provides an immune response and/or a therapeutic benefit in the treatment or management of a disease, condition, or disorder, or one or more symptoms associated therewith. The individual may be any mammal. In some implementations, the individual is human or an animal (e.g., a mammalian animal).

Routes of administration include all of those known in the art, including but not limited to, nasal, pulmonary, inhalation, injection, mucosal, oral, sublingual, gastrointestinal, transdermal, electrophoresis, intra-rectal, and intra-vaginal. In some implementations, routes of administration are non-invasive and pain-free as compared to injections. In one implementation, the composition is in liquid or solid form and administered orally. In some implementations, the methods and compositions involve oral, sublingual, and/or intranasal administration. As used herein, “oral” or “orally” relates to administration of the composition through the mouth and swallowed. Typical oral dosage forms include pills, tablets, capsules, sachets, solutions, emulsions, and syrups. In one embodiment, solid forms are preferred for ease of storage, shelf-life, and patient compliance. As used herein, “sublingual” or “sublingually” relates to administration of the composition underneath the tongue. A typical sublingual dosage form is a solid dosage form such as a lozenge put under the tongue where it dissolves and is presented to local tissue, but could also include a spray or other dosage form. As used herein, “intranasal” or “intranasally” relates to administration within the nose. A typical intranasal dosage form is an aerosol spray or liquid drop.

The doses can advantageously be administered by the individual or a trained clinician. Preferably, the doses are self-administered by the individual without assistance from a medical clinician. Although the pharmacological effect of a medication is a major factor in symptom management, clinical efficacy is dependent upon several additional factors. This may include the availability of an easy-to-swallow dosage formulation, such as a pill, caplet, capsule, granules, or a solution or suspension of an acceptable taste and texture compared to alternatives. In some implementations, the pharmaceutical compositions herein can be self-administered through methods of oral, sublingual, or intranasal routes, which allows for ease of administration of the pharmaceutical composition.

The treatment methods according to the present disclosure are effective in the treatment or management of a disease, condition, or disorder, or one or more symptoms thereof, including, but not limited to, leaky gut syndrome, Lyme disease, urinary tract infection, autoimmune disease, a coronavirus, influenza, blood infection, neurodegenerative disease, cancer, obesity, or any combination thereof. The treatment method may stimulate the individual's immune system, destroy or neutralize pathogens causing the disease, inhibit reproduction of pathogens causing the disease, or any combination thereof.

The dose of the mixture administered typically ranges from nanograms to milligrams of the mixture provided to the individual, depending on the mixture, the route of administration and the reactions of the individual's immune system. The dose is readily determined by one of ordinary skill in the art, particularly based on the guidance herein. In general, for a conventional administration route, the starting dose is typically one-half to double the maintenance dose. The use of an oral, sublingual, or nasal route of administration relative to injection typically allows for significantly higher cumulative doses over a shorter time period because of more frequent dosing that is allowed by self-administration and the safety profile afforded by these routes of administration.

In some implementations, the dosage may be set for an individual weighing up to 180 pounds, doubled for an individual weighing from greater than 180 to 250 pounds, and tripled for an individual weighing greater than 250 pounds. The set dosage may be, for example, about 10 to 1,000 mg, about 50 to 500 mg, about 100 to 400 mg, about 150 to 300 mg, or about 200 mg of the mixture. In such implementations, doses of a single, uniform specific concentration may be administered, without further dilution, using the weight-based dosage guidelines above. For example, in an oral dosing administration of a liquid pharmaceutical composition, the dose can include one or more drops of a liquid having a specific concentration of the mixture. For other dosage formulations, for example, quick dissolving tablets, a dose could be a quick dissolving tablet, and all of the tablets provided would have a uniform or specific concentration of the mixture. In one implementation, the concentration of mixture does not typically increase, but remains uniform or constant. Instead, the volume or the mass of the dosage form, e.g., a tablet, could increase or decrease to obtain the desired therapeutic amount at that time or after a given time interval, or an individual could take multiple tablets to obtain the desired concentration.

The dosing intervals can be anywhere between a few hours to a few days to one week to a few months over a course of a year. Typically, the dosing interval is no more than about one week, preferably no more than about 5 days, and more preferably no more than about one day. In exemplary implementations, the dosing interval is no more than about 12 hours, or no more than about 8 hours (i.e., 2-3 doses per day). The dosing intervals may be altered depending on the route of administration and the reactions of the individual's immune system.

In some implementations, the treatment method includes measuring a pre-treatment white blood cell count, which is elevated from normal for the individual due to the presence of a disease, condition, or disorder. During treatment, the white blood cell count may then be monitored and compared to the pre-treatment count as well as prior treatment counts. Treatment may then continue until the measured white blood cell count is either within normal ranges (e.g., below 13,000, 12,000, 11,000, or 1,0000 per μl of blood) or is reduced by a threshold percentage (e.g., at least 1%, at least about 5%, at least about 10%, or at least about 20%).

Greater concentrations of the mixture reduce the volume or mass of the dosage form administered, making the appropriate dose easier to administer. Doses can be driven to an even higher concentration without increasing the volume of pharmaceutical composition administered. For example, the volume of the pharmaceutical composition that is administered per day is typically about 0.1 ml to about 100 ml, about 0.5 ml to 50 ml, or about 1 ml to 30 ml when in liquid form. A dropper or volumetric pump can be made to deliver specific drop sizes, such as 0.1 ml or 1 ml. Similarly, solid dosage forms can have increasing amounts of the active pharmaceutical composition with fewer inert excipients in the carrier.

The methods for administration of the pharmaceutical compositions herein are not limited by the device used to deliver the compositions. Many devices are known in the art that can be used to deliver a known volume of a composition. For example, metered dose devices can be used to administer a liquid composition or an intranasal composition so that the amount of the mixture delivered to the individual increases with increasing volumes. An ideal device for sublingual or oral delivery consists of a pump system capable of delivering a precise dose of about 0.05 mL to 1 mL of the therapeutic composition and an actuator with an integrated spray or jet-stream insert. Alternatively, droppers can easily dispense the compositions of the present disclosure. Exemplary measured dosage devices can also include quick dissolving tablets or films, lozenges, infused papers, graduated measuring cups, or the like. Other routes of administration can employ devices known to those of ordinary skill in the art that are capable of delivering exact volumes of the compositions herein.

In one implementation, the pharmaceutical compositions are packaged in association with instructions on the treatment regimen. For example, the instructions can direct an individual to either administer the composition appropriately to the form, i.e., orally, sublingually, or intranasally, and then to repeat the administration at selected intervals, using appropriate doses of the pharmaceutical composition (e.g., same, higher or lower doses) with each successive dose. The therapeutic dose for a specific time interval, e.g., a day, a week, or a month, can be included in one or multiple packages or containers as discussed herein. For example, one package can include the initial treatment regimen with a dose for Day 1 and the days thereafter, where the doses are arranged if the dosage amounts differ over time (e.g., from a higher dose of the mixture to a lower dose if the first dose should be doubled), and then a second package with the maintenance treatment regimen for Day 2, 3, 4, 7, etc., for example, and the days or weeks thereafter, with the same size doses.

The present disclosure provides pharmaceutical compositions that include the mixture and a pharmaceutically acceptable carrier. The pharmaceutically acceptable carrier may include one or more excipients and adjuvants that aid in the administration discussed above. In some implementations, the pharmaceutical compositions may further include one or more additives, such as a solvent, humectant, emollient, plasticizer, a protein stabilizer, protease inhibitor, or bacteriostatic. In an implementation, the pharmaceutical composition comprises the TEW described above as a carrier or additive. In an implementation, the pharmaceutical composition comprises spring water or mineral water as a carrier. In an implementation, the pharmaceutical composition comprises glycerin. In some implementations, the carrier includes at least one of water, a buffer, saline, or a combination thereof. In one implementation, the carrier is present in a hypertonic concentration but is diluted at the time of dosing to create an isotonic, or slightly hypertonic solution for dosing by intranasal routes.

For an oral dosage, whether liquid or solid, the pharmaceutically acceptable carrier may include one or more optional additional additives to advantageously modify one or more formulation properties, including a pH modifying agent (e.g., buffering agent), stabilizing agent, flavoring agent, colorant agent, preservative agent, emulsifying agent, solubilizing agent, antioxidant agent, or any combination thereof. Any suitable selection or amount available to those of ordinary skill in the art may be included according to the disclosure so long as the carrier as a whole does not significantly detrimentally affect the stability of the mixture. Significantly detrimentally affecting stability might include reducing stability by more than 50 percent, or by reducing stability below a desired or required shelf-life, for example.

An oral pharmaceutical dosage form may also contain a flavoring agent. Any suitable flavoring agent available to those of ordinary skill in the art may be included in the composition, typically to enhance individual compliance by making the compositions more palatable. The flavoring agent is typically selected in type and amount to increase palatability, e.g., by decreasing or eliminating any undesired taste or off-flavors in the taste, i.e., a taste mask, that would otherwise be detectable by a typical individual to whom the pharmaceutical compositions are administered. Examples of a suitable flavoring agent, when used, include one or more natural or artificial flavorings, or both, including but not limited to one or more of menthol, peppermint, anise, and any fruit flavor, such as one or more of grapefruit, orange, banana, lemon, lime, mango, strawberry, pineapple, or cherry, natural and artificial fruit mix flavor, or a combination thereof.

Typical amounts of a flavoring agent, which is optional but preferred if the dosage is in liquid form, may be present in the carrier in an amount of about 0.05 percent (v/v) to 1.5 percent (v/v), based on the total volume of the composition. Exemplary amounts of flavoring agent can include about 0.2 percent (v/v) to 0.8 percent (v/v) or an amount of about 0.4 percent (v/v) to 0.6 percent (v/v), based on the total volume of the liquid composition.

A colorant agent, when included in the carrier, may be provided in an amount sufficient to provide the compositions with a more aesthetic and/or distinctive appearance. Any suitable colorant agent available to those of ordinary skill in the art may be selected. Typically, a colorant agent suitable for inclusion in the dosage forms includes one or more synthetic organic food additives (e.g., food dyes such as food red dye Nos. 2 and 3, food yellow dye Nos. 4 and 5 and food blue dye Nos. 1 and 2), water-insoluble lake dyes (e.g., aluminum salts of the above synthetic organic food additives, etc.), and natural pigments (e.g., beta-carotene, chlorophyll, iron oxide red, etc.). Other suitable colorants include D & C Red No. 33, FD & C Red No. 3, FD & C Red No. 40, D & C Yellow No. 10, and C Yellow No. 6, or any combination of these or the above colorants. In some implementations, only natural colorant agent materials are included.

A suitable preservative agent, if needed, may also be included in the carrier, typically in an amount sufficient to extend the shelf-life or storage stability, or both, of the pharmaceutical compositions. Examples of a suitable preservative agent, when used, include one or more of: sodium benzoate, paraoxybenzoic acid esters, methyl, ethyl, butyl, and propyl parabens, chlorobutanol, benzyl alcohol, phenylethylalcohol, dehydroacetic acid, sorbic acid, benzalkonium chloride (BKC), benzethonium chloride, phenylmercuric nitrate, thimerosal, or any combination thereof. A preservative agent may be added to the carrier at levels safe for ingestion. Typical amounts of preservative agent, when included in liquid form, may be from about 0.05 mg/5 mL to 10 mg/5 ml, based on the total volume of the solution. Exemplary amounts of preservative agent can include about 0.3 mg/5 mL to 5 mg/5 mL, based on the total volume of the oral liquid composition. Similar amounts may be used in other dosage forms.

Emulsifying agents can be used in the carrier in an amount sufficient to facilitate more uniform dispersion of the mixture or other excipient, preferably in liquid carriers for components that are not generally soluble in liquid form. Although any suitable emulsifying agent available to those of ordinary skill in the art can be used, if present, a preferred emulsifying agent includes gelatin, egg, casein, cholesterol, acacia, pectin, methyl cellulose, carbomer, cetostearyl alcohol, cetyl alcohol, or a combination thereof. In one implementation, the carrier is at least substantially free, or preferably entirely free of egg, alcohol, or both. It may be desirable to minimize or avoid these components because of the potential for allergic reaction in any individual having an allergy to eggs, and/or because of the potential burning or undesirable taste of these types of alcohol components.

Solubilizing agents can optionally be included, for example, in the carrier in an amount sufficient to facilitate greater or more rapid dissolution of the mixture or other excipient. Preferably, when included, the solubilizing agent is present in an amount sufficient to facilitate dissolving or dispersing the mixture or other therapeutically active components in the carrier. While any suitable solubilizing agent available to those of ordinary skill in the art can be included in the present formulations, preferably the solubilizing agent may include an alcohol, e.g., 95 percent ethyl alcohol, a glycol, glycerin, D-mannitol, trehalose, benzyl benzoate, trisaminomethane, cholesterol, triethanolamine, sodium carbonate, sodium citrate, sodium salicylate, sodium acetate, and a combination thereof. Preferred alcohols include ethanol, isopropanol, t-butanol, phenol, cresol, a benzyl alcohol, or a combination thereof. Preferably, the solubilizing agent may include a glycol. Suitable glycols may include, for example, those C2-20 alkenes functionalized with a glycol, including propylene glycol, polypropylene glycol, polyethylene glycol, etc., or a combination thereof. Preferred glycols include polyethylene glycol, such as PEG-400, or propylene glycol, or both. Typical amounts of solubilizing agent, when included, may be present in an amount of about 1 percent (v/v) to 20 percent (v/v), and more preferably about 4 percent (v/v) to 15 percent (v/v), based on the total volume of the oral liquid composition.

A stabilizing agent can include any suitable agent that increases the stability of the mixture. The stabilizing agent can include, for example, one or more liquid excipients such as ethanol, glycerin; one or more glycols, such as polyethylene glycol, e.g., PEG-400, propylene glycol, or polypropylene glycol; a cellulose-based component, such as hydroxypropylmethylcellulose (HPMC) or hydroxymethylcellulose (HMC); or any combination thereof. Thus, it should be understood that certain solubilizing agents may function effectively as a stabilizing agent. For example, propylene glycol may function as both a solubilizing agent and as a stabilizing agent.

Sublingual dosage forms, e.g., rapidly disintegrating tablets or quick dissolving films, are also well known to those of ordinary skill in the art, and typically include one or more of the following: disintegrants, binders, or lubricants, or any combination thereof. Preferred disintegrants include, but are not limited to, starches such as maize starch and rice starch, cross-linked N-vinyl-2-pyrrolidone (CLPVP), sodium starch glycolate, croscarmellose sodium and formaldehyde casein or combinations thereof. A preferred disintegrant is sodium starch glycolate. The disintegrant may be present as an intra-granular disintegrant or extra-granular disintegrant. The proportion of the disintegrant may be about 0.1 to 10 percent, preferably about 1 to 4 percent, and more preferably about 1.5 to 3 percent of the granule or other dosage form.

A binder may be employed, typically in solid dosage forms, in a minimum quantity to inhibit or prevent unnecessary reduction in the rate of dissolution. A preferred binder is polyvinyl pyrolidone or hydroxymethyl polyvinyl pyrolidone although others such as gelatin may also be used. Preferred binders are soluble in water when the binder is used in any liquid formulation (including a cream, emulsion, etc.).

Suitable lubricants for sublingual tablets include, but are not limited to, magnesium or calcium stearates or other long chain fatty acid salts. Magnesium stearate is especially preferred. A minimal proportion of lubricant is preferred, for example 0.1 percent up to about 1 percent, preferably about 0.8 percent. The lubricant may be an intra-granular lubricant, extra-granular lubricant or both. Any use of an extra-granular lubricant alone is preferred to minimize the hydrophobic properties of the dosage form.

The sublingual dosage form, e.g., tablet or film, may also include conventional excipients typically present at up to about 10 percent of the total weight. These may include flavoring agents, for example flavorings such as menthol, peppermint, vanilla or fruit flavorings. Flavoring agents when used are typically present up to about 0.5 to 5 percent by weight of the whole tablet. Further excipients may also include coloring agents, preservatives and fillers.

Preferred fillers can be selected from, for example without limitation, one or more saccharides. Mannitol, lactose, xylitol and mixtures thereof may be preferred in one implementation on account of their solubility and despite the water content of lactose in particular. Mannitol may be present in an amount of about 20 to 40 percent, for example about 20 to 30 percent by weight. Lactose may be present in an amount of about 30 to 60 percent, preferably about 45 to 60 percent by weight.

Intranasal sprays are also well known to those of ordinary skill in the art. These sprays typically include preservatives and tonicity adjusting agents. Common preservatives include one or more quaternary ammonium salts, such as lauralkonium chloride, benzalkonium chloride, benzododecinium chloride, cetyl pyridium chloride, cetrimide, domiphen bromide; alcohols such as benzyl alcohol, chlorobutanol, o-cresol, phenyl ethyl alcohol; organic acids or salts thereof such as benzoic acid, sodium benzoate, potassium sorbate, parabens; or complex forming agents such as EDTA; or any combination thereof. The amount of preservative may range from about 0.001 percent (w/w) to 0.1 percent (w/w). Glycerin, paraben, and/or sodium benzoate, are preferred preservatives, or preservative-free single dose aliquots may be prepared as well. Preferred compositions may include about 0.01 percent (w/w) of one or more preservatives.

Tonicity adjusting agents, such as sodium chloride, glucose, dextrose, mannitol, sorbitol, lactose and the like may also be added. Their amount is dependent upon the concentration of the other excipients. The tonicity of the composition may be approximately equal to the tonicity of blood. The bulk of the composition is water, preferably TEW disclosed herein or mineral water. Any tonicity adjusting agent, in a preferred implementation, is typically less than half formed, or at least substantially free, or entirely free, of any alcohol.

An important feature of an oral spray of an intranasal composition is its spray-ability, i.e., the ability of the composition to form an aerosol. This ability mainly depends upon the viscosity of the composition. When the composition is too viscous, the composition will not allow the formation of a spray. The composition will form large drops, or the composition may form a jet, when applying the spray device, thus resulting in a high concentration of active ingredient on a small area in the nasal cavity. Such high local concentration usually causes irritation. The composition should therefore have the right viscosity. It should be sprayed widely enough and so the droplets reside long enough in the nasal cavity to allow for sufficient bioavailability. The viscosity of the solution may range up to 50 mPa·s, although those of ordinary skill in the art will be readily able to determine suitable viscosity of the present compositions and for the present methods, particularly based on the guidance herein. The compositions may adhere to the mucosa, at least to some extent, and this may facilitate retention of the composition of the mucosa and/or enhance the absorption of the mixture. The compositions can be administered via the nasal route using a nasal spray device, pressurized aerosol canister or simple instillation means. To minimize or avoid overdosing and for hygienic reasons, a unidose nasal spray device is preferred.

The dosage form used to deliver the pharmaceutical compositions may be formulated for timed release, e.g. sustained or controlled release of the mixture over a selected period of time. Time-release technology allows for the convenience of administering a single dose, which releases an active ingredient over time, and may keep steadier levels of the ingredient in the blood stream rather than relying on a clinician or individual to administer additional doses at exact intervals. Sustained release and controlled release dosage forms can be formulated to release an increasing amount of mixture over an extended period of time so as to achieve a constant or stepwise rate of release of the mixture(s), which is particularly useful in the initial treatment phase of the methods herein. The increasing amounts of mixture correspond with therapeutically effective amounts of the mixture that reduces individual sensitivity to the mixture, and without increase in oral adverse effect, such as oral adverse event frequency. Any of the methods and materials available for formulating time release dosage forms are known by those having ordinary skill in the art and can be applied to the methods and compositions and dosage forms herein, particularly with guidance from the present specification. It should be understood as to all additives herein, whether in a sustained or controlled release form or not, that those reducing the conductivity of the water are less desired and should be minimized or avoided entirely.

In some implementations, the present disclosure provides for the use of any of the pharmaceutical compositions disclosed above in the preparation of a medicament for treatment of a disease, condition, or disorder in an individual in need. In some implementations, the present disclosure provides for the use of any of the pharmaceutical compositions disclosed above in therapy in an individual in need. In some implementations, the present disclosure provides for the use of any of the pharmaceutical compositions disclosed above in an effective dose for treating a disease, condition, or disorder in an individual in need. In some implementations, the present disclosure provides a kit for treating a disease, condition, or disorder in an individual in need comprising any of the pharmaceutical compositions disclosed above and instructions for use. In any of the foregoing implementations, the disease or disorder may include those described herein.

The term “about,” as used herein, should generally be understood to refer to both numbers in a range of numerals. Moreover, all numerical ranges herein should be understood to include each whole integer within the range where applicable, e.g., 35 to 65 would include 35, 36, 37, etc. as well as sub-ranges, e.g., 40 to 60, 45 to 55, 42 to 58, etc.

It is to be understood that the implementations are not limited to the particular systems or processes described, which may, of course, vary. It is also to be understood that the terminology used herein is for the purpose of describing particular implementations only and is not intended to be limiting. As used in this specification, the singular forms “a”, “an” and “the” include plural referents unless the context clearly indicates otherwise.

Although the present disclosure has been described in detail, it should be understood that various changes, substitutions, and alterations may be made herein without departing from the spirit and scope of the disclosure as defined by the appended claims. Moreover, the scope of the present application is not intended to be limited to the particular implementations of the process, machine, manufacture, composition of matter, means, methods, and steps described in the specification. As one of ordinary skill in the art will readily appreciate from the disclosure, processes, machines, manufacture, compositions of matter, means, methods, or steps, presently existing or later to be developed, that perform substantially the same function or achieve substantially the same result as the corresponding embodiments described herein may be utilized according to the present disclosure. Accordingly, the appended claims are intended to include within their scope such processes, machines, manufacture, compositions of matter, means, methods, or steps.

1. A pharmaceutical composition comprising:
structured particles comprising:
a ceramic substrate having a porous surface,
wherein the ceramic substrate comprises hydroxyl groups distributed over and associated with the porous surface;
titanium dioxide chemically bound to at least a portion of the porous surface; and
a metal or metal salt chemically bound to the titanium dioxide or the ceramic substrate; and
a pharmaceutically acceptable carrier,
wherein the structured particles are present in a pharmaceutically effective amount to provide a therapeutic effect.

2. The composition of claim 1, wherein the ceramic substrate comprises alumina, silica, zirconia, calcia, magnesia, ceria, sodia, potassia, lithia, boron oxide, manganese oxide, iron oxide, cobalt oxide, zinc oxide, or any combination thereof; and
wherein the metal or metal salt comprises silver, gold, iron, copper, platinum, palladium, cobalt, tin, zinc, zirconium, molybdenum, or any combination thereof.

3. The composition of claim 1, wherein the ceramic substrate comprises alumina, the metal or metal salt comprises a silver halide, and the pharmaceutically acceptable carrier comprises activated water having an electrical conductivity of at least 500 μS/cm at 25° C.

4. The composition of claim 1, further comprising a pharmaceutically acceptable additive comprising phosphorous, silica, alumina, boron, calcium, chromium, copper, iron, magnesium, manganese, potassium, gold, silver, sodium, sulfur, titanium, zinc, or any combination thereof.

5. The composition of claim 4, wherein a weight ratio of the structured particles to the additive is from about 1:1 to 1:20.

6. The composition of claim 1, wherein a weight ratio of the ceramic substrate to the titanium dioxide is from about 5:1 to 20:1; and
wherein a weight ratio of the ceramic substrate to the metal or metal salt is from about 5:1 to 20:1.

7. The composition of claim 1, wherein the structured particles are non-spherical and have an average particle size of least 0.1 microns to about 300 microns.

8. The composition of claim 1, wherein the structured particles are non-spherical and include a particle size of less than about 0.1 microns.

9. A method of making of the composition of claim 1, which comprises:
providing ceramic particles having an average particle size of about 3,000 microns;
forming a continuous layer of titanium dioxide on the ceramic particles;
binding the metal or metal salt to at least a portion of the continuous layer of titanium dioxide to form precursor particles;
micronizing the precursor particles to form the structured particles; and
dispersing the structured particles in the pharmaceutically acceptable carrier.

10. A pharmaceutical composition of claim 1, comprising the composition in a unit dose formula in a form selected from a solution, a suspension, an emulsion, a cream, a tablet, a capsule, a sachet, granules, a time-release formulation, an inhalant, or a nasal spray.

11. A dietary supplement composition comprising:
structured particles comprising:
a ceramic substrate having a porous surface;
titanium dioxide chemically bound to at least a portion of the porous surface; and
a metal or metal salt chemically bound to the titanium dioxide or the ceramic substrate; and
an electrically activated carrier;
wherein the structured particles are sized to be bioavailable.

12. The composition of claim 11, wherein the ceramic substrate comprises alumina, silica, zirconia, calcia, magnesia, ceria, sodia, potassia, lithia, boron oxide, manganese oxide, iron oxide, cobalt oxide, zinc oxide, or any combination thereof; and
wherein the metal or metal salt comprises silver, gold, iron, copper, platinum, palladium, cobalt, tin, zinc, zirconium, molybdenum, or any combination thereof.

13. The composition of claim 11, wherein the electrically activated carrier comprises activated water having an electrical conductivity of at least 500 μS/cm at 25° C.

14. The composition of claim 11, further comprising an additive comprising phosphorous, silica, alumina, boron, calcium, chromium, copper, iodine, iron, magnesium, manganese, molybdenum, selenium, potassium, silver, gold, sodium, sulfur, titanium, vanadium, zinc, lithium, carbon, chlorine, oxygen, nitrogen, hydrogen, bromine, alfalfa, chamomile, dandelion root, glutamic acid, horsetail, parsley, rice, watercress, yellow dock root, or any combination thereof.

15. The composition of claim 11, wherein the structured particles are non-spherical and have an average particle size of least 0.1 microns to about 300 microns.

16. The composition of claim 11, wherein the structured particles are non-spherical and include a particle size of less than about 0.1 microns.

17. A dietary supplement in dry powder form comprising the composition of claim 11 wherein the electrically activated carrier comprises rice powder infused with dehydrated electrically activated water.

18. A dietary supplement in drink form comprising one part by volume of the composition of claim 11 diluted with 30 parts by volume of drinking quality, spring water.

19. A method of making of the composition of claim 11, which comprises:
providing ceramic particles having an average particle size of about 3,000 microns;
forming a continuous layer of titanium dioxide on the ceramic particles;
binding the metal or metal salt to at least a portion of the continuous layer of titanium dioxide to form precursor particles;
micronizing the precursor particles to form the structured particles; and
dispersing the structured particles in the electrically activated carrier.

20. The method of claim 19 wherein the electrically activated carrier is formed by a method comprising:
treating water with a magnetic field by concentrating an electromagnetic spectrum of energy to the water.
